(19) United States
(12) Patent Application Publication
(10) Pub. No.: US 2017/0252446 A1
(43) Pub. Date: Sep. 7, 2017

(54) USE OF MESO- AND NANOPOROUS MATERIAL FOR SURFACTANT TRAPPING IN NANOPARTICLE SUSPENSIONS

(71) Applicants: Andreas VOIGT, Berlin (DE); Annette ASSOGBA-ZANDT, Berlin (DE); Christoph DUNMANN, Berlin (DE); Therakine BioDelivery GmbH, Berlin (DE)

(72) Inventors: Andreas Voigt, Berlin (DE); Annette Assogba-Zandt, Berlin (DE); Christoph Dunmann, Berlin (DE)

(21) Appl. No.: 15/517,972

(22) PCT Filed: Oct. 8, 2015
(86) PCT No.: PCT/US2015/054703

§ 371 Date: Apr. 10, 2017

Related U.S. Application Data
(60) Provisional application No. 62/061,733, filed on Oct. 9, 2014.

(51) Int. Cl. A61K 47/20; A61K 33/08; A61K 31/167; A61K 38/13; A61K 31/573; A61K 9/51; A61K 31/07; A61K 31/355; A61K 31/19; A61K 31/485; A61K 31/137; A61K 9/10; A61K 31/337

(52) U.S. Cl. CPC A61K 47/20; A61K 9/10; A61K 33/08; A61K 31/167; A61K 38/13; A61K 31/573; A61K 31/337; A61K 31/07; A61K 31/355; A61K 31/19; A61K 31/485; A61K 31/137; A61K 9/5192

(57) ABSTRACT

Disclosed are methods of making stable nanoparticle suspensions wherein one or more nanoporous or mesoporous materials are used to absorb and remove one or more non-tolerated surfactants from the nanoparticle suspensions. Also provided are methods of making stable nanoparticle suspension formulations wherein one or more macromolecular or colloidal stabilizers or tolerated surfactants are simultaneously added to further stabilize the nanoparticle suspension formulation. Thus to prevent a collapse of the suspension, one exchanges or replaces the surfactants that are not tolerated, by one or more tolerated surfactants or by macro-molecular/colloidal stabilizers.

### CROSS-REFERENCE TO RELATED APPLICATIONS

The present application is a national stage entry according to 35 U.S.C. §371 of PCT Application No. PCT/US2015/054703 filed on Oct. 8, 2015, which claims priority to U.S. Provisional Application Ser. No. 62/061,733 filed on Oct. 9, 2014.

### TECHNICAL FIELD

The subject matter herein generally relates to stabilizing nanoparticle suspensions where one or more nanoporous or mesoporous materials are used to adsorb or remove one or more non-tolerated surfactants from a nanoparticle suspension.

### BACKGROUND

Poorly soluble actives in pharmacy, cosmetics, and food industries need to have an increased bioavailability. Different methods have been used to achieve the increased bioavailability. For example, by increasing the interface area of the poorly soluble active in relation to the surrounding aqueous medium, the dissolution rate is increased. With conventional approaches of the past, no one has been able to create real stable nanoparticle suspensions of poorly soluble organic substances by a standard procedure. The present invention meets the long-felt need for stable nanoparticle suspensions of poorly soluble organic substances

### SUMMARY

According to certain embodiments, methods of making stable nanoparticle suspensions are provided, wherein a surfactant cocktail is used.

According to other embodiments, methods of making stable nanoparticle suspensions are provided, wherein one or more nanoporous or mesoporous materials are used to adsorb and remove one or more non-tolerated surfactants from the nanoparticle suspensions. According to yet another embodiment, a method of making stable nanoparticle suspension formulations is provided, wherein one or more macromolecular or colloidal stabilizers or tolerated surfactants are simultaneously added to further stabilize the nanoparticle suspension formulation. Thus, to prevent a collapse of the suspension, one exchanges or replaces the surfactants (that are not tolerated or permitted) by one or more tolerated surfactants or by macromolecular/colloidal stabilizers.

In a non-limiting embodiment, the one or more nanoporous or mesoporous materials will adsorb low-molecular weight surfactants but will not take up the macromolecular or colloidal stabilizers or the poorly soluble active ingredients or nanoparticles themselves. Mesoporous particles have pore sizes of a few nanometers only.

In a non-limiting embodiment, a method of making a stable nanoparticle suspension of a poorly soluble active ingredient comprises preparing the nanoparticle suspension; adding at least one surfactant to stabilize the nanoparticle suspension; and removal of essentially all non-tolerated surfactants from the nanoparticle suspension.

According to another embodiment, preparing the nanoparticle suspension comprises milling, precipitation, spray drying, or spray chilling.

According to yet another embodiment, adding the at least one surfactant comprises adding a surfactant cocktail to stabilize a high surface area of the nanoparticle suspension.

According to yet another embodiment, a maximum wetting of a poorly soluble material by an aqueous medium is achieved by the surfactant or surfactant cocktail.

According to yet another embodiment, to prevent a collapse of the suspension, one exchanges or replaces the one or more non-tolerated surfactants with one or more tolerated surfactants or macromolecular/colloidal stabilizers.

According to yet another embodiment, one or more macromolecular or colloidal stabilizers or tolerated surfactants are simultaneously added to further stabilize the nanoparticle suspension formulation.

According to yet another embodiment, the removal from the suspension of non-tolerated surfactants comprises adding one or more nanoporous or mesoporous materials to adsorb and remove one or more non-tolerated surfactants from the nanoparticle suspension.

According to yet another embodiment, the stable nanoparticle suspension comprises a plurality of anionic mesoporous nanoparticles.

According to yet another embodiment, the stable nanoparticle suspension comprises a plurality of cationic mesoporous nanoparticles.

According to yet another embodiment, the one or more nanoporous or mesoporous materials comprises at least one of neusilin, mesoporous materials MCM 41 (anionically and canonically equipped), titanium dioxide, silica gel, gamma aluminium oxide, bentonite, zeolite, calcium carbonate, or any combination thereof.

According to yet another embodiment, the one or more nanoporous or mesoporous materials adsorb low-molecular weight surfactants but not the macromolecular or colloidal stabilizers or the poorly soluble active ingredient.

According to yet another embodiment, the one or more mesoporous materials comprise mesoporous structures having pore sizes ranging from about 2 nm to about 50 nm.

According to yet another embodiment, the poorly soluble active ingredient is selected from the group consisting of acyclovir, acrivastine, aceclofenac, acetaminophen (i.e., paracetamol), adriamycin, albendazole, acetazolamide, acetylsalicylic acid, albuterol, allopurinol, amlodipine, amoxicillin, amphetamine, azathioprine, azelastine, amphoteticin B, angiotensin converting enzyme (ACE) or NEP inhibitors, atorvastatin, allopurinol, 1-carbocysteine, aluminium hydroxide, amoxicillin, atovaquone, azithromycin, baclofen, benidipine, bicalutamide, busulfan, bisacodyl, cabergoline, butenafine, calcipotdene, calcitriol, camptothecin, cannabinoids, capsaicin, carbamazepine, carotenes, cefdinir, cefditoren pivoxil, cefixime, celecoxib, cerivastatin, cefotiam hexetil hydrochloride, cefpodoxime proxetil, cefuroxime axetil, cetirizine, candesartan cilexetil, chloroquine, chlorpromazine, cilostazol, carvedilol, chlorpheniramine, cimetidine, ciprofloxacin, cisapride, clarithromycin, clemastine, codeine, cyclosporine, clofazimine, clopidogrel, clozapine, cyproterone, dapsone, danazol, dantrolene, dexchlorpheniramine, dexamethasone, digoxin, dirithromycin, donepezil, dexamethasone, diazepam, diclofenac, diloxanide, doxycycline, ebastine, efavirenz, eprosartan and other sartans, epalrestat, ergotamines, esomeprazole, estrogens, etodolac, etoposide, erythromycin ethylsuccinate, ethyl icosapentate, ezetimibe, famotidine, fenofibrate, fibric acid derivatives, fentanyl, fexofenadine, finasteride, fluconazole, flurbiprofen, flutamide, fluvastatin, fosphenytoin, frovatriptan, famotidine, folic acid, furosemide, gabapentin, gemfibrozil, glibenclamide, glimepiride, gefitinib, gliclazide, glipizide, glyburide, griseofulvin, glibenclamide, haloperidol, hydrochlorothiazide, hydroxyzine, halofantrine, ibuprofen, pralnacasan, indomethacin, irinotecan, imatinib, indinavir, iopanoic acid, irbesartan, isotretinoin, isradipine, itraconazole, ivermectin, ketoconazole, ketoprofen, ketorolac, lamotrigine, lansoprazole, leflunomide, levodopa, levosulpiride, linezolid, loperamide, lopinavir, loratadine, lovastatin, lorazepam, lycopenes, manidipine, mebendazole, mefloquine, medroxyprogesterone, melphalan, meloxicam, mesalamine, menatetrenone, metaxalone, methadone, methoxsalen, metoclopramide, metronidazole, miconazole, midazolam, mifepristone, miglitol, mitoxantrone, modafinil, methylphenidate, mosapride, mycophenolate mofetil, nabumetone, nalbuphine, nalidixic acid, naproxen, naratriptan, nelfinavir, nevirapine, nicergoline, niclosamide, nifedipine, nilutamide, nizatidine, nilvadipine, nimesulide, nitrofurantoin, nystatin, olanzapine, orlistat, omeprazole, oxaprozin, oxcarbazepine, oxycodone, paclitaxel, pentazocine, phenytoin, phenobarbital, pioglitazone, pizotifen, pralnacasan, pranlukast, praziquantel, propylthiouracil, pravastatin, probucol, pyrantel, pyrimethamine, lansoprazole, pseudoephedrine, propylthiouracil, pyridostigmine, quetiapine, quinine, rabeprazole, raloxifene, repaglinide, rebamipide, retinol, rifampicin, rifapentine, rimexolone, risperidone, ritonavir, rizatriptan, rofecoxib, rosiglitazone, saquinavir, sibutramine, roxithromycin, sennosides, sildenafil citrate, simvastatin, sirolimus, spironolactone, steroids, sulfadiazine, sulfamethoxazole, sulfasalazine, sultamicillin, sumatriptan, tacrine, tacrolimus, tamoxifen, telmisartan, teprenone, tamsulosin, targretin, tazarotene, teniposide, terbinafine, testosterones, tiagabine, ticlopidine, tocopherol nicotinate, tizanidine, theophylline, topiramate, topotecan, valproic acid, valsartan, toremifene, tosufloxacin, tramadol, tretinoin, trimethoprim, triflusal, troglitazone, trovafloxacin, verapamil, warfarin, ursodeoxycholic acid, verteporfin, vigabatrin, vitamin A, vitamin D, vitamin E, vitamin K, zaltoprofen, zafirlukast, zileuton, zolmitriptan, zolpidem, zopiclone, or pharmaceutically acceptable salts,isomers, prodrugs and derivatives thereof, and any combination thereof.

In a non-limiting environment, one can utilize a micronization method using surfactants in one or another form.

According to yet another embodiment, bioavailability of the poorly soluble active ingredient is increased with the stable nanoparticle suspension.

### DETAILED DESCRIPTION

The following language and descriptions of certain non-limiting embodiments of the present invention are provided to further an understanding of the principles of the present invention. However, it will be understood that no limitations of the present invention are intended, and that further alterations, modifications, and applications of the principles of the present invention are also included.

According to the present invention, the methods of preparing stable nanoparticle suspensions can be utilized as a reliable and promising approach for increasing the bioavailability of poorly soluble active ingredients. To achieve this, according to certain embodiments, top down (milling) as well as bottom up (precipitation) technologies can be used. In other words, in certain embodiments, nanoparticle preparation can include milling, and another method involves precipitation from solution. An appropriate surfactant cocktail is needed to stabilize the high surface area of a nanoparticle suspension and achieve a successful nanoparticle suspension formulation. A maximum wetting of the poorly soluble active ingredient by an aqueous medium is achieved by the surfactant or surfactant cocktail. To achieve the optimum surfactant cocktail, it is desired to achieve the smallest possible contact angle of the surfactant or surfactant cocktail solution at the poorly soluble active material surface.

However, the surfactants or surfactant cocktail which have to be used for an optimum result are not tolerated or permitted by pharmaceutical, cosmetic, or food regulatory authorities (such as the FDA). The vast majority of surfactants are rather toxic and have to be removed/replaced by non-toxic molecular species before application, for example, in pharmacy. Therefore, according to the present invention, methods are provided for the removal of the surfactants or surfactant cocktail that are not tolerated or permitted by pharmaceutical, cosmetic, or food regulatory authorities. According to the methods of the present invention, after obtaining an optimum nanoparticle size suspension, surfactants or surfactant cocktail that are not tolerated or permitted are essentially removed from the nanoparticle suspension.

As used herein, “adsorption” refers to the adhesion or accumulation of molecules of gas, liquid, or dissolved solids on the surface of a solid or liquid.

As used herein, “microporous” refers to a porous structure defined by pores having a diameter of less than about 2 nanometers (nm).

As used herein, “mesoporous” refers to a porous structure defined by pores having a pore diameter of only about a few nanometers. Non-limiting mesoporous structures may have pore sizes from about 2 nm to about 50 nm.

As used herein, “macroporous” refers to a porous structure defined by pores having a diameter of greater than about 50 nm.

As used herein, “colloid” refers to a homogeneous, noncrystalline substance consisting of large molecules or ultramicroscopic particles of a substance dispersed through and suspended in a medium. Colloids can include, for example, gels, sols, and emulsions.

As used herein, “macromolecule” is intended to include a molecule of high relative molecular mass, the structure of which essentially comprises the multiple repetition of units derived, actually or conceptually, from molecules of low relative molecular mass. “Macromolecule” can include, for instance, nucleic acids, proteins, and carbohydrates, as well as non-polymeric molecules with large molecular mass such as, for instance, lipids and macrocycles.

As used herein, a “poorly soluble” active ingredient is intended to include, for example, any active ingredient that is either sparingly soluble, slightly soluble, poorly soluble, very slightly soluble, has a minimal solubility in water, has a low solubility or that is insoluble in water. The solubility can be increased by decreasing the particle sizes of the poorly soluble active ingredients. The solubility increases when the poorly soluble active ingredients are at or below 100 nm.

Examples of such “poorly soluble” active ingredients include, but are not limited to, acyclovir, acrivastine, aceclofenac, acetaminophen (i.e., paracetamol), adriamycin, albendazole, acetazolamide, acetylsalicylic acid, albuterol, allopurinol, amlodipine, amoxicillin, amphetamine, azathioprine, azelastine, amphotericin B, angiotensin converting enzyme (ACE) or NEP inhibitors, atorvastatin, allopurinol, 1-carbocysteine, aluminium hydroxide, amoxicillin, atovaquone, azithromycin, baclofen, benidipine, bicalutamide, busulfan, bisacodyl, cabergoline, butenafine, calcipotriene, calcitriol, camptothecin, cannabinoids, capsaicin, carbamazepine, carotenes, cefdinir, cefditoren pivoxil, cefixime, celecoxib, cerivastatin, cefotiam hexetil hydrochloride, cefpodoxime proxetil, cefuroxime axetil, cetirizine, candesartan cilexetil, chloroquine, chlorpromazine, cilostazol, carvedilol, chlorpheniramine, cimetidine, ciprofloxacin, cisapride, clarithromycin, clemastine, codeine, cyclosporine, clofazimine, clopidogrel, clozapine, cyproterone, dapsone, danazol, dantrolene, dexchlorpheniramine, dexamethasone, digoxin, dirithromycin, donepezil, dexamethasone, diazepam, diclofenac, diloxanide, doxycycline, ebastine, efavirenz, eprosartan and other sartans, epalrestat, ergotamines, esomeprazole, estrogens, etodolac, etoposide, erythromycin ethylsuccinate, ethyl icosapentate, ezetimibe, famotidine, fenofibrate, fibric acid derivatives, fentanyl, fexofenadine, finasteride, fluconazole, flurbiprofen, flutamide, fluvastatin, fosphenytoin, frovatriptan, famotidine, folic acid, furosemide, gabapentin, gemfibrozil, glibenclamide, glimepiride, gefitinib, gliclazide, glipizide, glyburide, griseofulvin, glibenclamide, haloperidol, hydrochlorothiazide, hydroxyzine, halofantrine, ibuprofen, pralnacasan, indomethacin, irinotecan, imatinib, indinavir, iopanoic acid, irbesartan, isotretinoin, isradipine, itraconazole, ivermectin, ketoconazole, ketoprofen, ketorolac, lamotrigine, lansoprazole, leflunomide, levodopa, levosulpiride, linezolid, loperamide, lopinavir, loratadine, lovastatin, lorazepam, lycopenes, manidipine, mebendazole, mefloquine, medroxyprogesterone, melphalan, meloxicam, mesalamine, menatetrenone, metaxalone, methadone, methoxsalen, metoclopramide, metronidazole, miconazole, midazolam, mifepristone, miglitol, mitoxantrone, modafinil, methylphenidate, mosapride, mycophenolate mofetil, nabumetone, nalbuphine, nalidixic acid, naproxen, naratriptan, nelfinavir, nevirapine, nicergoline, niclosamide, nifedipine, nilutamide, nizatidine, nilvadipine, nimesulide, nitrofurantoin, nystatin, olanzapine, orlistat, omeprazole, oxaprozin, oxcarbazepine, oxycodone, paclitaxel, pentazocine, phenytoin, phenobarbital, pioglitazone, pizotifen, pralnacasan, pranlukast, praziquantel, propylthiouracil, pravastatin, probucol, pyrantel, pyrimethamine, lansoprazole, pseudoephedrine, propylthiouracil, pyridostigmine, quetiapine, quinine, rabeprazole, raloxifene, repaglinide, rebamipide, retinol, rifampicin, rifapentine, rimexolone, risperidone, ritonavir, rizatriptan, rofecoxib, rosiglitazone, saquinavir, sibutramine, roxithromycin, sennosides, sildenafil citrate, simvastatin, sirolimus, spironolactone, steroids, sulfadiazine, sulfamethoxazole, sulfasalazine, sultamicillin, sumatriptan, tacrine, tacrolimus, tamoxifen, telmisartan, teprenone, tamsulosin, targretin, tazarotene, teniposide, terbinafine, testosterones, tiagabine, ticlopidine, tocopherol nicotinate, tizanidine, theophylline, topiramate, topotecan, valproic acid, valsartan, toremifene, tosufloxacin, tramadol, tretinoin, trimethoprim, triflusal, troglitazone, trovafloxacin, verapamil, warfarin, ursodeoxycholic acid, verteporfin, vigabatrin, vitamin A, vitamin D, vitamin E, vitamin K, zaltoprofen, zafirlukast, zileuton, zolmitriptan, zolpidem, zopiclone, or pharmaceutically acceptable salts, isomers, prodrugs and derivatives thereof, and any combination thereof. One of skill in the art will recognize additional suitable active ingredients not described herein.

As used herein, “stable” is intended to refer to, for example, a nanoparticle suspension that has increased stability that is achieved based on the methods as described herein.

As used herein, “surfactant” refers to any substance or compound which lowers the surface tension (or interfacial tension) between two liquids or between a liquid and a solid, thereby increasing its spreading and wetting properties.

As used herein, a “surfactant” that is “permitted” or “tolerated” is a surfactant that is essentially non-toxic or harmless. Representative examples of tolerated or permitted surfactants can include, for instance, any permitted and suitable cationic, anionic, zwitterionic and non-ionic surfactants that are essentially non-toxic or harmless, e.g., as determined based on safety standards recognized by the pharmaceutical industry and appropriate regulatory authorities, e.g., the Food and Drug Administration (FDA). One of skill in the art will recognize that any suitable non-toxic or harmless surfactants can be used.

According to an embodiment, a method of making stable nanoparticle suspensions is provided, wherein a surfactant cocktail is used.

According to another embodiment, a method of making stable nanoparticle suspensions is provided, wherein one or more nanoporous or mesoporous materials are used to adsorb and remove one or more non-tolerated surfactants from the nanoparticle suspensions.

In one embodiment, if one runs several cycles of addition and removal of the mesoporous material, one can effectively and essentially remove the toxic surfactants (which had to be used essentially for the nanoparticle preparation process itself) while performing simultaneous addition of harmless surfactants. These harmless (but permitted and non-toxic) surfactants are surprisingly useful for stabilizing the nanoparticle system. This stabilization is needed for a limited time period, e.g., only up to the final formulation of the nanoparticles into medication/application forms (tablets, gels etc.).

According to yet another embodiment, a method of making stable nanoparticle suspension formulations is provided, wherein one or more macromolecular or colloidal stabilizers or tolerated (harmless) surfactants are simultaneously added to further stabilize the nanoparticle suspension formulation.

In a non-limiting embodiment, the one or more nanoporous or mesoporous materials will adsorb the low-molecular weight surfactants but will not take up the macromolecular or colloidal stabilizers or the poorly soluble active ingredients or nanoparticles themselves because both are too large for that purpose. Mesoporous particles have pore sizes of a few nanometers only.

One can use any suitable surfactant or surfactant cocktail for the nanoparticle preparation process. It is understood that the suitable surfactant or surfactant cocktail is adequate and compatible with the technical process. In at least one embodiment, the technical process can be, but is not limited to, milling, precipitation, spray drying, or spray chilling.

It is to be understood that other suitable surfactants or surfactant cocktails (that can be tolerated) may be used.

After nanoparticle preparation, essentially all surfactants and/or surfactant cocktails (that are not tolerated or permitted) may be removed and replaced with one or more suitable stabilizers of the nanoparticle suspension. Such suitable stabilizers of the nanoparticle suspension may include, for example, suitable permitted and tolerated surfactants or polymers, like gelatin, etc. Removal of the surfactants and/or surfactant cocktails (that are not tolerated or permitted) is achieved by using one or more nanoporous or mesoporous materials.

In at least one embodiment, the nanoporous or mesoporous material can include, for example, but is not limited to, neusilin, mesoporous materials MCM 41 (anionically and cationically equipped), titanium dioxide, silica gel, gamma aluminium oxide, bentonite, zeolite, or calcium carbonate. It is to be understood that other suitable nanoporous or mesoporous materials may be used.

### Potential Applications

The following are some examples of potential applications. These applications are not limiting, and are for illustrative purposes only. The methods can be used, for example, in the preparation of poorly soluble active ingredient nanoparticle suspensions for pharmacy, cosmetics, and food comprising only permitted passive ingredients and nanoparticles of only a few hundred nanometers and smaller. In accordance with a non-limiting embodiment, a large surface area is presented by nanoparticles to guarantee a sufficient dissolution velocity or increased solubility.

The methods can also be used for efficient removal of surfactant or adsorbable low-molecular weight ingredients from any technical system by not acting on systems component larger than the pore size.

### EXAMPLES

The present embodiments are further illustrated by the following examples. It is to be understood that the following examples shall not limit the scope of the invention in any way.

### Example 1

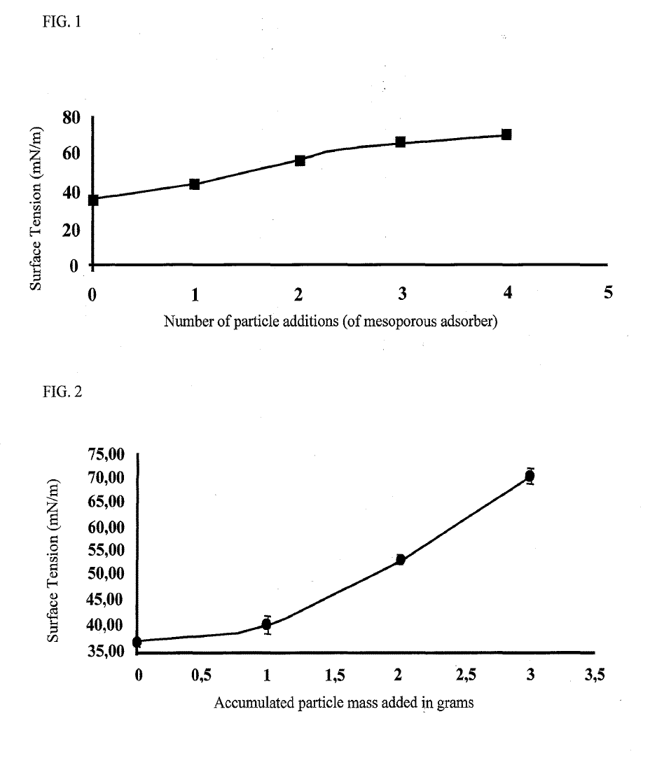

Anionic mesoporous nanoparticles (MCM) were suspended into water, wetted, and separated from free water by centrifugation. Pure sodium dodecyl sulphate (SDS) solution (8.2 mM) provides a starting surface tension of about 36.5 mN/m. Per 1 g of MCM particles, 15 mL of SDS is added, resulting in an increase of the surface tension to 45 mN/m. Second, third, and fourth additions result in 57.5 mN/m, 66.8 mN/m, and 70.5 mN/m, respectively. After four mesoporous particle additions, the pure water value is already approached. This is illustrated in FIG. 1. Such non-limiting methods have been unexpectedly discovered to be useful for reliably and predictably preparing a stable nanoparticle suspension of a poorly soluble active ingredient.

### Example 2

Cationic mesoporous nanoparticles (MCM) are suspended in water, wetted, and separated from free water by centrifugation. Pure sodium dodecyl sulphate (SDS) solution (8.2 mM) provides a starting surface tension of about 36.5 mN/m. Per 1 g of MCM particles, 15 mL of SDS is added, resulting in an increase of the surface tension to 40 mN/m. Second and third additions result in 54.0 mN/m and 70.0 mN/m, respectively. After three mesoporous particle additions, the pure water value is already approached. This is illustrated in FIG. 2. Such non-limiting methods have been unexpectedly discovered, according to the present invention, to be useful for reliably and predictably preparing a stable nanoparticle suspension of a poorly soluble active ingredient.

The embodiments shown and described above are only examples. Even though numerous characteristics and advantages of the present technology have been set forth in the foregoing description, it is to be understood that the disclosure is illustrative only.

What is claimed is:

1. A method of making a stable nanoparticle suspension of a poorly soluble active ingredient, comprising:
preparing the nanoparticle suspension;
adding at least one surfactant to stabilize the nanoparticle suspension; and
removing essentially all non-tolerated surfactants from the nanoparticle suspension.

2. The method of claim 1, wherein preparing the nanoparticle suspension comprises milling, precipitation, spray drying, spray chilling, and combinations thereof.

3. The method of claim 1, wherein adding the at least one surfactant comprises adding a surfactant cocktail to stabilize a high surface area of the nanoparticle suspension.

4. The method of claim 1, wherein a maximum wetting of a poorly soluble material by an aqueous medium is achieved by the surfactant or surfactant cocktail.

5. The method of claim 1, wherein to prevent a collapse of the suspension, the one or more non-tolerated surfactants is exchanged or replaced with one or more tolerated surfactants or macromolecular/colloidal stabilizers.

6. The method of claim 1, wherein one or more macromolecular or colloidal stabilizers or tolerated surfactants are simultaneously added to further stabilize the nanoparticle suspension formulation.

7. The method of claim 1, wherein the removal from the suspension of non-tolerated surfactants comprises adding one or more nanoporous or mesoporous materials to adsorb and remove one or more non-tolerated surfactants from the nanoparticle suspension.

8. The method of claim 7, wherein the stable nanoparticle suspension comprises a plurality of anionic mesoporous nanoparticles.

9. The method of claim 7, wherein the stable nanoparticle suspension comprises a plurality of cationic mesoporous nanoparticles.

10. The method of claim 7, wherein the one or more nanoporous or mesoporous materials comprises at least one of neusilin, mesoporous materials MCM 41 (anionically and cationically equipped), titanium dioxide, silica gel, gamma aluminium oxide, bentonite, zeolite, calcium carbonate, or any combination thereof.

11. The method of claim 7, wherein the one or more nanoporous or mesoporous materials adsorb low-molecular weight surfactants but not the macromolecular or colloidal stabilizers or the poorly soluble active ingredient.

12. The method of claim 7, wherein the one or more mesoporous materials comprise mesoporous structures having pore sizes ranging from about 2 nm to about 50 nm.

13. The method of claim 1, wherein the poorly soluble active ingredient is selected from the group consisting of acyclovir, acrivastine, aceclofenac, acetaminophen paracetamol), adriamycin, albendazole, acetazolamide, acetylsalicylic acid, albuterol, allopurinol, amlodipine, amoxicillin, amphetamine, azathioprine, azelastine, amphotericin B, angiotensin converting enzyme (ACE) or NET inhibitors, atorvastatin, allopurinol, 1-carbocysteine, aluminium hydroxide, amoxicillin, atovaquone, azithromycin, baclofen, benidipine, bicalutamide, busulfan, bisacodyl, cabergoline, butenafine, calcipotriene, calcitriol, camptothecin, cannabinoids, capsaicin, carbamazepine, carotenes, cefdinir, cefditoren pivoxil, cefixime, celecoxib, cerivastatin, cefotiam hexetil hydrochloride, cefpodoxime proxetil, cefuroxime axetil, cetirizine, candesartan cilexetil, chloroquine, chlorpromazine, cilostazol, carvedilol, chlorpheniramine, cimetidine, ciprofloxacin, cisapride, clarithromycin, clemastine, codeine, cyclosporine, clofazimine, clopidogrel, clozapine, cyproterone, dapsone, danazol, dantrolene, dexchlorpheniramine, dexamethasone, digoxin, dirithromycin, donepezil, dexamethasone, diazepam, diclofenac, diloxanide, doxycycline, ebastine, efavirenz, eprosartan and other sartans, epalrestat, ergotamines, esomeprazole, estrogens, etodolac, etoposide, erythromycin ethylsuccinate, ethyl icosapentate, ezetimibe, famotidine, fenofibrate, fibric acid derivatives, fentanyl, fexofenadine, finasteride, fluconazole, flurbiprofen, flutamide, fluvastatin, fosphenytoin, frovatriptan, famotidine, folic acid, furosemide, gabapentin, gemfibrozil, glibenclamide, glimepiride, gefitinib, gliclazide, glipizide, glyburide, griseofulvin, glibenclamide, haloperidol, hydrochlorothiazide, hydroxyzine, halofantrine, ibuprofen, pralnacasan, indomethacin, irinotecan, imatinib, indinavir, iopanoic acid, irbesartan, isotretinoin, isradipine, itraconazole, ivermectin, ketoconazole, ketoprofen, ketorolac, lamotrigine, lansoprazole, leflunomide, levodopa, levosulpiride, linezolid, loperamide, lopinavir, loratadine, lovastatin, lorazepam, lycopenes, manidipine, mebendazole, mefloquine, medroxyprogesterone, melphalan, meloxicam, mesalamine, menatetrenone, metaxalone, methadone, methoxsalen, metoclopramide, metronidazole, miconazole, midazolam, mifepristone, miglitol, mitoxantrone, modafinil, methylphenidate, mosapride, mycophenolate mofetil, nabumetone, nalbuphine, nalidixic acid, naproxen, naratriptan, nelfinavir, nevirapine, nicergoline, niclosamide, nifedipine, nilutamide, nizatidine, nilvadipine, nimesulide, nitrofurantoin, nystatin, olanzapine, orlistat, omeprazole, oxaprozin, oxcarbazepine, oxycodone, paclitaxel, pentazocine, phenytoin, phenobarbital, pioglitazone, pizotifen, pralnacasan, pranlukast, praziquantel, propylthiouracil, pravastatin, probucol, pyrantel, pyrimethamine, lansoprazole, pseudoephedrine, propylthiouracil, pyridostigmine, quetiapine, quinine, rabeprazole, raloxifene, repaglinide, rebamipide, retinol, rifampicin, rifapentine, rimexolone, risperidone, ritonavir, rizatriptan, rofecoxib, rosiglitazone, saquinavir, sibutramine, roxithromycin, sennosides, sildenafil citrate, simvastatin, sirolimus, spironolactone, steroids, sulfadiazine, sulfamethoxazole, sulfasalazine, sultamicillin sumatriptan, tacrine, tacrolimus, tamoxifen, telmisartan, teprenone, tamsulosin, targretin, tazarotene, teniposide, terbinafine, testosterones, tiagabine, ticlopidine, tocopherol nicotinate, tizanidine, theophylline, topiramate, topotecan, valproic acid, valsartan, toremifene, tosufloxacin, tramadol, tretinoin, trimethoprim, triflusal, troglitazone, trovafloxacin, verapamil, warfarin, ursodeoxycholic acid, verteporfin, vigabatrin, vitamin A, vitamin D, vitamin E, vitamin K, zaltoprofen, zafirlukast, zileuton, zolmitriptan, zolpidem, zopiclone, or pharmaceutically acceptable salts, isomers, prodrugs and derivatives thereof, and any combination thereof.

14. The method of 3, wherein a bioavailability of the poorly soluble active ingredient is increased with the stable nanoparticle suspension.
